(19) United States
(12) Patent Application Publication
(10) Pub. No.: US 2009/0105497 A1
(43) Pub. Date: Apr. 23, 2009

(54) Reduced Hazard Thermal Fluid

(75) Inventors: Manuel Medrano Rodriguez, Cd. Juarez (MX); Carlos Roberto Cuellar Gurrola, Cd. Juarez (MX); Alejandro Lopez Ortiz, Chihuahua (MX); Virginia Hidolina Collins Martinez, Chihuahua (MX); Manuel David Delgado Vigil, Cuauhtemoc (MX)

Correspondence Address: MCNEES WALLACE & NURICK LLC, 100 PINE ST., P.O. BOX 1166, HARRISBURG, PA, 17108-1166, US

(73) Assignee: JOHNSON CONTROLS TECHNOLOGY COMPANY, Holland, MI (US)

(21) Appl. No.: 11/877,113

(22) Filed: Oct. 23, 2007

(51) Int. Cl. C07C 69/00; G05D 23/02

(52) U.S. Cl. 560/129; 236/93 R; 73/204.22

(57) ABSTRACT

A thermal fluid having a fluid that includes a compound selected from the group consisting of compounds having the following general formulas:

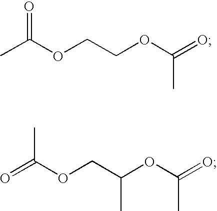

and combinations thereof is provided.

The fluid is arranged and disposed to expand or contract in response to temperature. A temperature sensing element and a method for sensing a temperature are also disclosed.

### BACKGROUND

The application generally relates to liquid compositions for use in thermal sensing equipment. The application relates more specifically to fluids that can substitute toluene in thermal sensing equipment utilized for Heating, Ventilation, Air Conditioning and Refrigeration (HVAC&R) systems.

Liquid toluene, which has a known coefficient of expansion, is a thermal fluid utilized to fill temperature sensing elements and temperature sensing equipment. Toluene has been considered a hazardous material by the U.S. Department of Transportation and has been classified by National Fire Protection Association (NFPA) within the NFPA fire diamond as having a Health Value of 2, Flammability Value of 4 and Reactivity Value of 1. This last classification includes an extremely hazardous flammability and a moderately hazardous health hazard requiring human protection through the use of a breathing apparatus. The use of fluids having such classifications are undesirable in temperature sensing equipment.

A temperature sensing element is a closed piece of metal tubing filled with a liquid. The tubing is configured with one of the ends of the element including a flexible diaphragm that exhibits contraction or expansion as a result of a corresponding volume change of the liquid that can be induced by a temperature change of the element. The original volume of the liquid contained in the element is known at a given temperature and the liquid has a known coefficient of thermal expansion. In other words, since the volume and coefficient of expansion of the liquid is known, the expansion and contraction of the liquid (and hence the diaphragm) can be determined as the temperature changes. The expansion and contraction follows the following formula:

ΔV=β×Vᵢ×ΔT

Where ΔV corresponds to the volume change of the liquid in cubic inches (in³), β represents the coefficient of thermal expansion of the fluid inverse degree Fahrenheit (1/° F.) Vᵢ is the initial volume at the initial temperature in cubic inches (in³) and ΔT is temperature change in degrees Fahrenheit (° F.). The lower value of β of a fluid, the greater temperature change required to produce a given diaphragm movement. Therefore, if the value of β for a given liquid is smaller than the value of β for toluene, a different and larger range of differential movement of the diaphragm for the same volume of liquid and the same diaphragm configuration of the element is expected for the given liquid as compared to toluene. Toluene has been used in various temperature sensing elements and temperature sensing equipment for at least 50 years. Existing thermal fluids known as being less hazardous than toluene using the NFPA classification, are typically synthetic, organic, low viscosity materials having a β smaller than toluene. Such differences in β of the fluids require a redesign of the thermal sensing equipment including an increased bulb size and/or larger differentials in the specification for the element if the other fluid is to be used.

Intended advantages of the disclosed systems and/or methods satisfy one or more of these needs or provide other advantageous features. Other features and advantages will be made apparent from the present specification. The teachings disclosed extend to those embodiments that fall within the scope of the claims, regardless of whether they accomplish one or more of the aforementioned needs.

### SUMMARY

One embodiment relates to a thermal fluid having a fluid that includes a compound selected from the group consisting of compounds having the following general formulas:

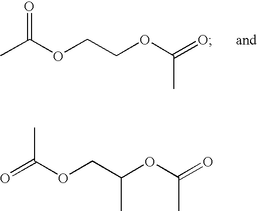

Combinations of the fluids may also be utilized. The fluid is arranged and disposed to expand or contract in response to temperature.

Another embodiment relates to a temperature sensing element including a variable volume chamber. A fluid is disposed within the chamber. The fluid includes a compound selected from the group consisting of compounds having the following general formulas:

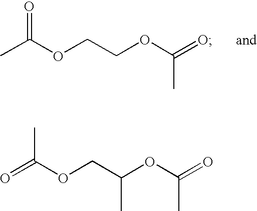

Combinations of the fluids may also be utilized. The fluid is arranged and disposed to expand or contract in response to a temperature change. The expansion or contraction of the fluid within the chamber varies the volume of the chamber.

Still another embodiment relates to a method for sensing a temperature condition. The method includes providing a variable volume chamber. The method further includes a fluid within the chamber, where the fluid includes a compound selected from the group consisting of compounds having the following general formulas:

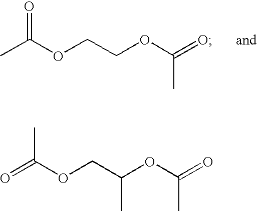

Combinations of the fluids may also be utilized. The fluid is arranged and disposed to expand or contract in response to a temperature change. The method further includes exposing the element to a temperature change. The volume of the chamber is monitored and a temperature is determined from the volume of the chamber.

Certain advantages of the embodiments described herein are a reduced hazard fluid, including reduced toxicity, flammability and reductions in other hazards with respect to toluene.

In addition, the thermal fluids are capable of replacing current thermal fluids without significant changes in design or alternation of existing equipment.

Alternative exemplary embodiments relate to other features and combinations of features as may be generally recited in the claims.

### BRIEF DESCRIPTION OF THE FIGURES

FIG. 1 is a temperature sensing element according to an embodiment.

FIG. 2 is a volumetric device according to an embodiment.

FIG. 3 shows data of volumetric change over temperature ranges of a fluid according to an embodiment.

### DETAILED DESCRIPTION OF THE EXEMPLARY EMBODIMENTS

The thermal fluids or temperature sensing fluids according to one embodiment include ethylene glycol diacetate (CH₃COOCH₂CH₂OOCCH₃), also known as 1,2-ethanediol diacetate, ethylene diacetate, 1,2-diacetoxyethane and/or 2-(acetyloxy)ethyl acetate and propylene glycol diacetate (CH₃CH(OCOCH₃)CH₂OCOCH₃), also known as 1,2-propanediol diacetate, propylene diacetate, 1,2-diacetoxypropane and/or 2-(acetyloxy)-1-methylethyl acetate. Specifically, the thermal fluids according to the embodiment includes compositions having a compound selected from the group consisting of compounds having the following general formulas:

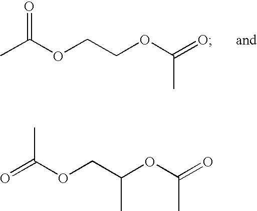

Combinations of the fluids may also be utilized.

The thermal fluids used in temperature sensing elements of the one embodiment include fluids having a β and thermal characteristics substantially identical to toluene. Table 1 describes values for β for toluene as well as for the two thermal fluids in a temperature range of −58 degrees F. to 392 degrees F.

TABLE 1
β VALUES FOR TOLUENE AND REPLACEMENT FLUIDS
Toluene β (degrees Fahrenheit⁻¹ (° F.⁻¹)), (degrees Celsius⁻¹ (° C.⁻¹)):

| | |
|---|---|
| 0.000600-0.000630 (−58 to 32 degrees Fahrenheit⁻¹ (° F.)) | 0.00134-0.00140 (−50 to 0 degrees Celsius⁻¹ (° C.)) |
| 0.000630-0.000664 (32 to 122 degrees Fahrenheit⁻¹ (° F.)) | 0.00140-0.00148 (0 to 50 degrees Celsius⁻¹ (° C.)) |
| 0.000664-0.000706 (122 to 212 degrees Fahrenheit⁻¹ (° F.)) | 0.00148-0.00157 (50 to 100 degrees Celsius⁻¹ (° C.)) |
| 0.000706-0.000758 (212 to 302 degrees Fahrenheit⁻¹ (° F.)) | 0.00157-0.00169 (100 to 150 degrees Celsius⁻¹ (° C.)) |
| 0.000758-0.000828 (302 to 392 degrees Fahrenheit⁻¹ (° F.)) | 0.00169-0.00184 (150 to 200 degrees Celsius⁻¹ (° C.)) |

Ethylene glycol diacetate β (degrees Fahrenheit−¹ (° F.⁻¹)), (degrees Celsius−¹ (° C.⁻¹))
Thermal Fluid #1:

| | |
|---|---|
| 0.000529-0.000553 (−58 to 32 degrees Fahrenheit−¹ (° F.)) | 0.00098-0.00103 (−50 to 0 degrees Celsius⁻¹ (° C.)) |
| 0.000553-0.000580 (32 to 122 degrees Fahrenheit−¹ (° F.)) | 0.00103-0.00108 (0 to 50 degrees Celsius⁻¹ (° C.)) |
| 0.000580-0.000610 (122 to 212 degrees Fahrenheit−¹ (° F.)) | 0.00108-0.00114 (50 to 100 degrees Celsius⁻¹ (° C.)) |
| 0.000610-0.000649 (212 to 302 degrees Fahrenheit−¹ (° F.)) | 0.00114-0.00121 (100 to 150 degrees Celsius⁻¹ (° C.)) |
| 0.000649-0.000697 (302 to 392 degrees Fahrenheit−¹ (° F.)) | 0.00121-0.00130 (150 to 200 degrees Celsius⁻¹ (° C.)) |

Propylene glycol diacetate β (degrees Fahrenheit⁻¹ (° F.⁻¹)), (degrees Celsius−¹ (° C.⁻¹))
Thermal Fluid #2:

| | |
|---|---|
| 0.000580-0.000606 (−58 to 32 degrees Fahrenheit⁻¹ (° F.)) | 0.00108-0.00113 (−50 to 0 degrees Celsius⁻¹ (° C.)) |
| 0.000606-0.000635 (32 to 122 degrees Fahrenheit⁻¹ (° F.)) | 0.00113-0.00118 (0 to 50 degrees Celsius⁻¹ (° C.)) |
| 0.000635-0.000670 (122 to 212 degrees Fahrenheit⁻¹ (° F.)) | 0.00118-0.00125 (50 to 100 degrees Celsius⁻¹ (° C.)) |
| 0.000670-0.000711 (212 to 302 degrees Fahrenheit⁻¹ (° F.)) | 0.00125-0.00132 (100 to 150 degrees Celsius⁻¹ (° C.)) |
| 0.000711-0.000763 (302 to 392 degrees Fahrenheit⁻¹ (° F.)) | 0.00132-0.00142 (150 to 200 degrees Celsius⁻¹ (° C.)) |

The thermal fluids #1 and #2 have an average coefficient of thermal expansion of from about 0.00098 inverse degrees Celsius (° C.⁻¹) to about 0.00130 inverse degrees Celsius (° C.⁻¹) (Thermal Fluid #1) or from about 0.00108 inverse degrees Celsius (° C.⁻¹) to about 0.00142 inverse degrees Celsius (° C.⁻¹) (Thermal Fluid #2) at operational temperatures. Operational temperatures include those temperatures for which the HVAC & R equipment may be exposed. Operational temperature include a range from about −58 degrees Fahrenheit (° F.) to about 392 degrees Fahrenheit (° F.) (−50 degrees Celsius (° C.) to about 200 degrees Celsius (° C.)).

Thermal fluids #1 and #2 have not been classified as hazardous materials by the U.S. Department of Transportation and include a NFPA classification within the NFPA fire diamond as having Health Values of 1, Flammability Values of 2 and Reactivity Values of 0. The thermal fluid according to the one embodiment are less hazardous than toluene. By less hazardous, the thermal fluids have lower flammability than toluene and/or have a lower health risk than toluene.

TABLE 2
SUBSTANCE IDENTIFICATION

| | | THERMAL | THERMAL |
|---|---|---|---|
| CHARACTERISTIC | TOLUENE | FLUID #1 | FLUID #2 |
| Fluid known as: | Methylbenzene, | 1,2-Ethanediol, | 1,2-Propanediol, |
| | Toluol or | diacetate, Ethylene | diacetate, |
| | Phenylmetane | glycol diacetate, | Propylene glycol |
| | | Ethylene diacetate, | diacetate, |
| | | 1,2- | Propylene |
| | | Diacetoxyethane, | diacetate, |
| | | 2-(Acetyloxy)ethyl | 1,2- |
| | | acetate | Diacetoxypropane, |
| | | | 2-(Acetyloxy)-1- |
| | | | methylethyl acetate |
| | | | 2-(Acetyloxy)ethyl |
| | | | acetate |
| Formula | C₆H₅CH₃ | C₆H₁₀O₄ | C₇H₁₂O₄ |
| CAS Number | 108-88-3 | 111-55-7 | 623-84-7 |
| Appearance | Clear, colorless | Clear, colorless | Colorless liquid |
| | liquid (Saybolt +28-+30) | liquid (APHA 15 | |
| | | Max) | |
| Odor | Aromatic, benzene- | Mild, pleasant | Mild, pleasant |
| | like | | |

TABLE 3
PHYSICAL AND CHEMICAL PROPERTIES

| | | THERMAL | THERMAL |
|---|---|---|---|
| CHARACTERISTIC | TOLUENE | FLUID #1 | FLUID #2 |
| Molecular Weight | 92.15 | 146.12 | 160.17 |
| Boiling Point | 111° C. | 186-193° C. | 190-195° C. |
| @ 760 mm Hg | (232° F.) | (366.8-373.3° F.) | (374-379.4° F.) |
| Melting Point | −95° C. | −40.9-−30.9° C. | −38.9-−30.9° C. |
| | (−139° F.) | (−42.7-−23.6° F.) | (−38.0-−23.6° F.) |
| Specific Gravity | 0.86 | 1.106 ± 0.002 | 1.059 ± 0.002 |
| @ 20/20° C./TD | | | |
| Vapor Density (vs air) | 3.14 | 5.04 | 5.52 |
| Vapor Pressure | 22.0 | 0.24 | 0.57 |
| @ 20 C (68° F.) Torr | | | |
| Evaporation Rate | 2.24 | 0.02 | 0.02 |
| (butyl acetate = 1): | | | |
| Flash Point | 4° C. | 88-96.1° C. | 87° C. |
| | (39.2° F.) | (190-209.9° F.) | (188° F.) |
| | | (Cleveland open | |
| | | cup) | |
| Solubility in Water | 0.5 | 160 | 77 |
| @ 20° C. (68° F.) g/l | (0.05 gm/100 gm | | |
| | water) | | |
| Dynamic Viscosity | 0.55 | 2.9 | 3.0 |
| @ 25° C. cP | | | |
| Viscosity @ 20° C. cSt | 0.68 | 2.58 | 2.83 |
| Surface Tension | 28.6 | 20 | — |
| (mN/m) | | | |
| Refractive Index @ | 1.4933 | 1.4159 | 1.414 |
| 20° C. | | | |
| Acidity, as Acetic | No Free acid | 0.05% Max | — |
| Acid, WT % (ASTM | (Neutral) | | |
| D-847) | | | |
| Chemical Stability | Stable under | Stable under | Stable under |
| | ordinary conditions | ordinary conditions | ordinary conditions |
| Autoignition | 480° C. | 481° C. | 481° C. |
| Temperature | (896° F.) | (897.80° F.) | (897.80° F.) |

TABLE 4
REGULATORY INFORMATION

| | | THERMAL | THERMAL |
|---|---|---|---|
| CHARACTERISTIC | TOLUENE | FLUID #1 | FLUID #2 |
| DOT | Hazard Class 3, PG | Not Listed | Not Listed |
| | II, Flammable | | |
| | Liquid | | |
| UN Class | UN1294, | Not Listed | Not Listed |
| Air Transport | No exceptions for | No exceptions for | No exceptions for |
| | air transportation | air transportation | air transportation |
| Passenger | Quantity Limit is 5 L. | Not Listed | Not Listed |
| Aircraft/Rail | | | |
| Cargo Aircraft | Quantity Limit is | Not Listed | Not Listed |
| | 60 L | | |
| NFPA Health | 2 | 1 | 1 |
| NFPA Fire | 4 | 2 | 2 |
| NFPA Reactivity | 1 | 0 | 0 |

TABLE 5
HEALTH HAZARD INFORMATION

| | | THERMAL | THERMAL |
|---|---|---|---|
| CHARACTERISTIC | TOLUENE | FLUID #1 | FLUID #2 |
| Respiratory PPE | Chemical Cartridge | OSHA 29, CFR | OSHA 29, CFR |
| | Air Respirator | 1910.134 | 1910.134 |
| Skin Protection PPE | Viton or | Neoprene Gloves | Wear suitable |
| | Viton/Neoprene | | protective clothing |
| | Gloves | | to prevent contact |
| | | | with skin |
| Eye Protection PPE | Safety glasses with | Safety glasses with | OSHA 29, CFR |
| | side shields | side shields | 1910.133 |
| Threshold Limit | 100 | — | — |
| Values (TLV) ppm (8 h | | | |
| NIOSH) | | | |

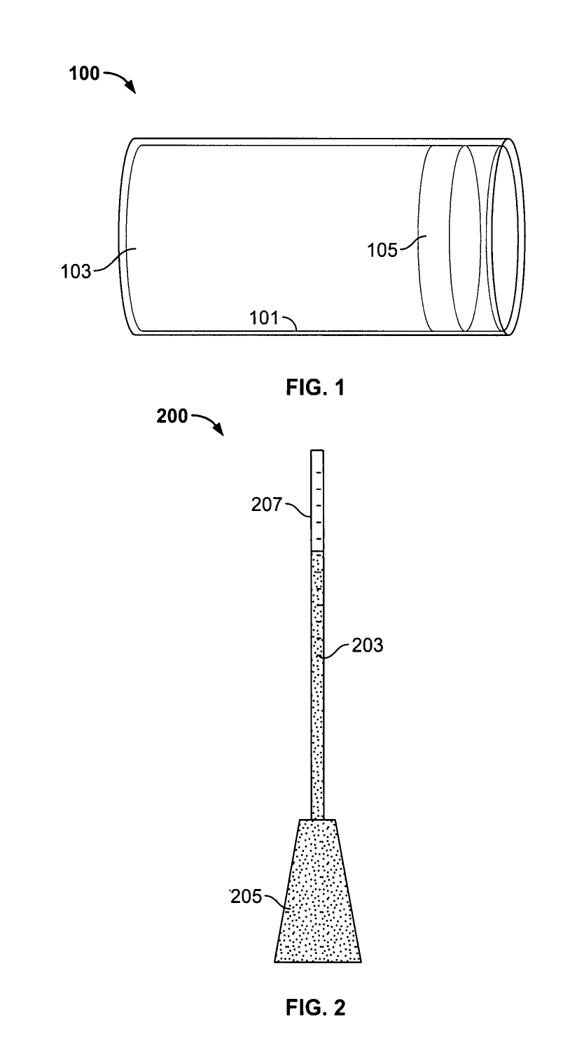

FIG. 1 shows a temperature sensing element 100 for use with a component of an HVAC&R system or other system. The temperature sensing element 100 determines the temperature of a fluid or other medium in response to the expansion of a fluid 103 contained within the temperature sensing element 100. The temperature sensing element 100 may be a component of the control system or other equipment and provide sensing and/or control for the components of the system. The temperature sensing element 100 includes a chamber 101 configured to receive a fluid 103. The chamber 101 includes a variable volume. The volume may be varied by a movable side-wall 105, which is capable of moving in response to expansion or contraction of the fluid 103. The chamber 101 and side-wall 105 are fabricated from a resilient material or elastic material having a low coefficient of thermal expansion. The geometry of the chamber 101 is not limited to a cylindrical geometry and may include any geometry capable of receiving a fluid 103. In addition, the side-wall 105 is not limited to the arrangement shown and may include any structure that provide variable volume to chamber 101. Fluid 103 includes thermal fluids having compositions including a compound selected from the group consisting of compounds having the following general formulas:

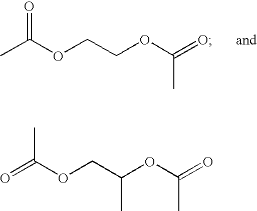

Combinations of the fluids may also be utilized.

In operation, the temperature sensing element 100 is exposed to a temperature. For example, a medium whose temperature is sensed may be provided in close proximity to the temperature sensing element 100. In addition, portions of the chamber 101 or extensions thereto may be fabricated from a thermally conductive material in contact or in close proximity to the fluid or other medium to be measured. For example, outside air or interior air may be sensed and the temperature may be determined, wherein the temperature sensing element 100 may be in contact with the air, in close proximity to the air, or a component or extension of the temperature sensing element 100 may be in contact or close proximity to the air. In response to the exposure to the medium temperatures, fluid 103 expands or contracts and the volume of chamber 101 is increased or decreased accordingly. The volume of the chamber 101 is measured and the temperature is calculated based upon the volume resulting from the exposure to the medium. The temperature can be calculated, with respect to a reference temperature, wherein the change in volume is measured from the following formula:

ΔV=β×Vᵢ×ΔT

Calibrations based upon volumes of a known fluid or other known techniques may be utilized to provide temperature measurements with respect to a reference temperature. Likewise, differential temperatures may be measured and determined with respect to a difference in changes in volume of the thermal fluid.

### EXAMPLE

The following example illustrates the similar behavior between toluene and thermal fluid #1. The test includes filling a volumetric device 200 with a predetermined volume of test fluid 203 at a predetermined temperature, wherein one device 200 is filled with toluene and another with thermal fluid #1, as shown in FIG. 2. Specifically, 68 mL of thermal fluid #1 was added to the device 200 at 140° F. The device 200 is composed of a glass reservoir 205 and graduated volumetric tube 207. When the fluid filled device 200 is exposed to temperature changes, the expansion thermal coefficient of the test fluid 203 produces corresponding volume changes. Throughout the experiment, changes in temperature with respect to volume are recorded and presented in Table 2.

TABLE 6
Experimental data

| | THERMAL | | | THERMAL | |
|---|---|---|---|---|---|
| TEMPERATURE | FLUID #1 | TOLUENE | TEMPERATURE | FLUID #1 | TOLUENE |
| (° F.) | (ml) | (ml) | (° F.) | (ml) | (ml) |
| 140 | 68.0 | 68.0 | −30 | 62.1 | 61.9 |
| 130 | 67.7 | 67.7 | −20 | 62.1 | 62.0 |
| 120 | 67.3 | 67.3 | −10 | 62.4 | 62.4 |
| 110 | 66.9 | 66.9 | 0 | 62.8 | 62.8 |
| 100 | 66.6 | 66.5 | 10 | 63.2 | 63.1 |
| 90 | 66.2 | 66.1 | 20 | 63.5 | 63.5 |
| 80 | 65.8 | 65.7 | 30 | 63.9 | 63.8 |
| 70 | 65.5 | 65.4 | 40 | 64.3 | 64.2 |
| 60 | 65.2 | 65.1 | 50 | 64.6 | 64.5 |
| 50 | 64.9 | 64.7 | 60 | 65.0 | 64.9 |
| 40 | 64.5 | 64.3 | 70 | 65.4 | 65.2 |
| 30 | 64.1 | 64.0 | 80 | 65.7 | 65.6 |
| 20 | 63.7 | 63.5 | 90 | 66.0 | 65.9 |
| 10 | 63.5 | 63.3 | 100 | 66.5 | 66.4 |
| 0 | 63.1 | 62.9 | 122 | 67.3 | 67.2 |
| −10 | 62.7 | 62.5 | 130 | 67.5 | 67.5 |
| −20 | 62.4 | 62.2 | 140 | 67.9 | 68.0 |
| −30 | 62.1 | 61.9 | | | |

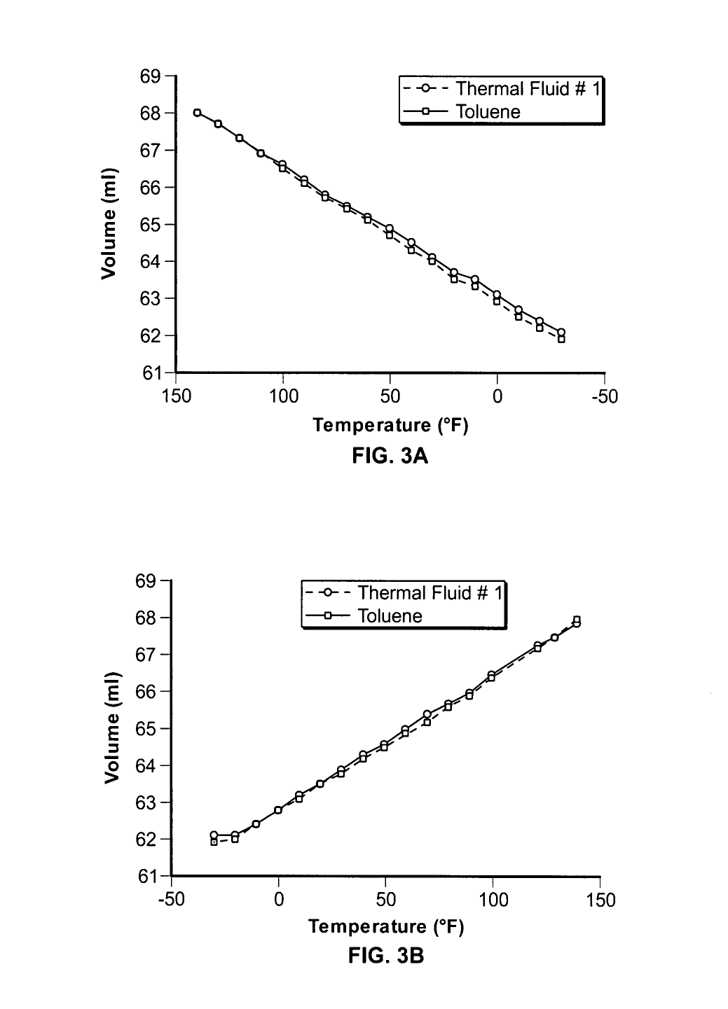

Experimental data resulting from measurements taken as the test fluid 203 is exposed to the changing temperatures is plotted in FIGS. 3a and 3b. In these figures, temperature decrease is plotted in FIG. 3A and temperature increase is plotted in FIG. 3B. From both plots, it is evident that the thermal behavior of the thermal fluid is substantially identical to the thermal behavior of toluene.

It should be understood that the application is not limited to the details or methodology set forth in the following description or illustrated in the figures. It should also be understood that the phraseology and terminology employed herein is for the purpose of description only and should not be regarded as limiting.

While the exemplary embodiments illustrated in the figures and described herein are presently preferred, it should be understood that these embodiments are offered by way of example only. Accordingly, the present application is not limited to a particular embodiment, but extends to various modifications that nevertheless fall within the scope of the appended claims. The order or sequence of any processes or method steps may be varied or re-sequenced according to alternative embodiments.

It is important to note that the construction and arrangement of the apparatus as shown in the various exemplary embodiments is illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited in the claims. For example, elements shown as integrally formed may be constructed of multiple parts or elements, the position of elements may be reversed or otherwise varied, and the nature or number of discrete elements or positions may be altered or varied. Accordingly, all such modifications are intended to be included within the scope of the present application. The order or sequence of any process or method steps may be varied or re-sequenced according to alternative embodiments. In the claims, any means-plus-function clause is intended to cover the structures described herein as performing the recited function and not only structural equivalents but also equivalent structures. Other substitutions, modifications, changes and omissions may be made in the design, operating conditions and arrangement of the exemplary embodiments without departing from the scope of the present application.

It should be noted that although the figures and specification herein may show and/or describe a specific order of method steps, it is understood that the order of these steps may differ from what is depicted. Also two or more steps may be performed concurrently or with partial concurrence. Such variation will depend on the software and hardware systems chosen and on designer choice. It is understood that all such variations are within the scope of the application. Likewise, software implementations could be accomplished with standard programming techniques with rule based logic and other logic to accomplish the various connection steps, processing steps, comparison steps and decision steps.

What is claimed is:

1. A thermal fluid comprising:
a fluid including a compound selected from the group consisting of compounds having the following general formulas: 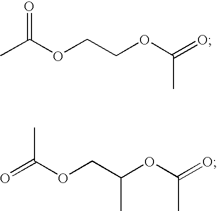
and combinations thereof,
wherein the fluids are arranged and disposed to expand or contract in response to temperature.

2. The thermal fluid of claim 1, wherein the fluid has an average coefficient of thermal expansion of from about 0.00098° C.-1 to about 0.00130 at operational temperatures.

3. The thermal fluid of claim 1, wherein the fluid has an average coefficient of thermal expansion of from about 0.00108° C.-1 to about 0.00142 at operational temperatures.

4. The thermal fluid of claim 1, wherein the fluid has a lower flammability than toluene.

5. The thermal fluid of claim 1, wherein the fluid has a lower toxicity than toluene.

6. The thermal fluid of claim 1, wherein the fluid has a lower flammability and lower toxicity than toluene.

7. A temperature sensing element for use in an HVAC & R system comprising:
a variable volume chamber,
a fluid disposed within the chamber, the fluid including a compound selected from the group consisting of compounds having the following general formulas: 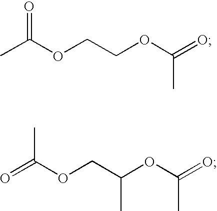
and combinations thereof,
wherein the fluid is arranged and disposed to expand or contract in response to temperature, wherein expansion or contraction of the fluid within the chamber varies the volume of the chamber.

8. The temperature sensing element of claim 7, wherein the fluid has a coefficient of thermal expansion of from about 0.00098° C.⁻¹ to about 0.00130 at operational temperatures.

9. The temperature sensing element of claim 7, wherein the fluid has a coefficient of thermal expansion of from about 0.00108° C.⁻¹ to about 0.00142 at operational temperatures.

10. The temperature sensing element of claim 7, wherein the fluid has a lower flammability than toluene.

11. The temperature sensing element of claim 7, wherein the fluid has a lower toxicity than toluene.

12. The temperature sensing element of claim 7, wherein the element senses a temperature value according to the expansion or contraction of the fluid.

13. A method for sensing a temperature condition comprising:
providing a variable volume chamber,
providing a fluid within the chamber, the fluid including a compound selected from the group consisting of compounds having the following general formulas: 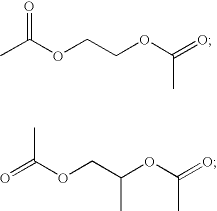
and combinations thereof,
wherein the fluid is arranged and disposed to expand or contract in response to temperature,
exposing the element to temperature;
monitoring the volume of the chamber; and
determining a temperature from the volume of the chamber.

14. The method of claim 13, wherein the fluid has a coefficient of thermal expansion of 0.00098° C.⁻¹ to about 0.00130 at operational temperatures.

15. The method of claim 13, wherein the fluid has a coefficient of thermal expansion of 0.00108° C.⁻¹ to about 0.00142 at operational temperatures.

16. The method of claim 13, wherein the fluid has a lower flammability than toluene.

17. The method of claim 13, wherein the fluid has a lower toxicity than toluene.

18. The method of claim 13, wherein the fluid has a lower flammability and lower toxicity than toluene.

19. The method of claim 13, further comprising operating an HVAC component in response to a temperature determined in the determining step.
